(19) United States
(12) Patent Application Publication
(10) Pub. No.: US 2026/0207930 A1
(43) Pub. Date: Jul. 23, 2026

(54) CUTTING HELIX FOR LEAD FIXATION

(71) Applicant: SORIN CRM SAS, CLAMART (FR)

(72) Inventors: JEAN-FRANÇOIS OLLIVIER, SAINT REMY LES CHEVREUSES (FR); VINCENT CHOUPEAUX, LES ALLEUTS LE ROI (FR)

(21) Appl. No.: 19/134,994

(22) PCT Filed: Dec. 2, 2022
(86) PCT No.: PCT/IB2022/000694

§ 371 Date: Jun. 2, 2025

(51) Int. Cl. A61N 1/05

(52) U.S. Cl. CPC A61N 1/0573

(57) ABSTRACT

The invention relates to a lead device that comprises a lead tip with a distal fixation helix for fixing the lead tip at a patient's tissue (e.g., cardiac tissue, in particular tissue of the septum) wherein the fixation helix comprises a cutting portion for cutting the tissue in a circumferential direction of the fixation helix, in addition to a puncturing by a distal tip of the fixation helix (30), when the fixation helix is screwed into the tissue, to support longitudinal insertion of the lead tip with the fixation helix into the tissue. Thereby, insertion and longitudinal advancement of the lead tip into the tissue can be facilitated and smoothened due to a pre-cutting of the tissue in a circumferential direction at and/or around and/or within the fixation helix during the screwing operation into the tissue.

### FIELD OF THE INVENTION

The invention relates to the field of fixation structures for lead devices (e.g., electrode catheters) for cardiac pacing systems, such as—but not limited to—left bundle branch pacing (LBBP), cardiac resynchronization or tachycardia (“tachy”) systems.

### BACKGROUND OF THE INVENTION

Different electrical activation sequences of cardiac pacing may lead to different mechanical pump efficiencies of a stimulated heart. What is needed is a fast and homogenous contraction of heart ventricles to optimize pump efficiency.

Traditional pacing sites such as the right ventricular apex (RVA) may provide a stable lead position with low displacement rate but are not very effective to optimize left ventricle (LV) contraction (representing about 80% of the heart mass). Long-term RVA pacing may have deleterious effects on left ventricular function by inducing an iatrogenic left bundle branch block (LBBB), which can have strong influences on the left ventricle hemodynamic performances. This observation led to a reassessment of traditional approaches and to a research of alternative pacing sites, in order to get to more physiological pattern of ventricular activation and to avoid deleterious effects.

LBBP has emerged as an alternative method for delivering physiological pacing to achieve electrical synchrony of the LV, especially in patients with infranodal atrioventricular block and/or LBBB. The proximal LBBs run through the LV septum and fan out to form a wider target for pacing compared to the His bundle. A technique for LBBP has been developed using a ventricular transseptal approach (i.e., pacing the LV from the RV). LBBP has been reported to offer low pacing thresholds and large R waves, and because the distal conduction system is targeted, has a lower theoretical risk for development of distal conduction block.

After an initial site for an LBBP location at the right surface of the ventricular septum has been determined, the pacing lead (i.e., a helical fixation element or electrode at the lead tip) is screwed into the LV septum, e.g., by puncturing the tissue with the distal tip of the helical fixation element (fixation helix). The LBBP lead depth into the LV septum may be determined by at least one of observing changes in the notch in V1 lead, sheath angiography, fulcrum sign, and impedance monitoring. The pacing lead is slowly progressed into the determined depth (e.g., approximately 6 to 8 mm) by the application of a torque, meanwhile avoiding any perforation of the septum. Finally, LBB capture is confirmed based on acceptable pacing parameters. The confirmation may be based on at least one of a paced morphology of an RBBB pattern, a recording of an LBB potential, a stimulus-peak of the LVAT that shortens abruptly with increasing output or remains shortest and constant at low and high outputs, a selective LBBP and a non-selective LBBP, and a recording of a retrograde His potential or anterograde LBB potential during pacing.

Common features of implantation or placement processes include transvenous access, transseptal placement of the pacing lead into the LV septal subendocardium in the LBB region, and confirmation of capture of the LBB.

The tips of pacing or tachy leads are typically designed to avoid a risk of perforation of the septum. They may also be equipped with a soft tip (made of e.g. Silicone) to increase a stop surface. That is, when the helical fixation element or electrode (called “helix” hereinafter) is engaged with (e.g., screwed into) the (cardiac) issue, this tissue is pushed against the soft tip to stop the helix from rotation and further progression within the tissue. The length of the helix may be limited, e.g., to an active length of about 2 mm.

However, a challenge concerning the puncturing process at the septum or other tissue resides in an optimization of the progression performance of the helix with respect to the required energy/force for puncturing and the design of the lead to allow a well-controlled and safe progression without increasing complexity of the lead. To this end, it needs to be born in mind that the inner surface of the cavities of the RV and LV are “covered” with a thin robust skin (membrane or lining) named the “endothelium”, that is much more tougher to puncture than the inner section of the septum.

### SUMMARY OF THE INVENTION

It is an object of the present invention to provide an electrode catheter system that addresses the above challenges faced in connection with LBBP or other pacing approaches and enhance efficiency of the puncturing process.

This object is achieved by a fixation element as claimed in claim 1.

The proposed lead device comprises a lead tip with a distal fixation helix for fixing the lead tip at a patient's tissue (e.g., cardiac tissue, in particular tissue of the septum) wherein the fixation helix comprises a cutting portion for cutting the tissue in a circumferential (circular) direction of the fixation helix in addition to a puncturing by a distal tip of the fixation helix, when the fixation helix is screwed into the tissue, to support longitudinal insertion of the lead tip with the fixation helix into the tissue.

Accordingly, insertion and longitudinal advancement (motricity) of the lead tip into the tissue can be facilitated and smoothened due to a pre-cutting of the tissue in a circumferential direction at and/or around and/or within the fixation helix during the screwing operation into the tissue.

According to a first option, the cutting portion may comprise two neighboring turns of the fixation helix with a closed intermediate gap in the longitudinal direction between the two neighboring turns. During the screwing motion of the fixation helix into the tissue, the tissue is strongly compressed between the two neighboring turns with closed gap, which results in a scissor-like cutting effect in the circumferential direction of the fixation helix. Thereby, a simple and cost-efficient implementation of the additional cutting effect is obtained.

According to a second option, which can be combined with the first option, the two neighboring turns of the fixation helix may be provided in a proximal portion of the fixation helix. Thereby, the distal portion of the fixation helix serves to assist the longitudinal advancement of the lead device via the screwing operation.

According to a third option, which can be combined with the first or second option, at least one of the two neighboring turns of the fixation helix may comprise a cross-sectional shape with a sharp edge towards the opposing neighboring turn. Thus, the sharpened edge(s) leads to an increased pressure on the tissue and a better cutting effect.

According to a fourth option, which can be combined with any of the first to third options, a conical insert may be fixed at the distal end of the lead tip and surrounded by the fixation helix. The conical insert presses the tissue within the fixation helix outwards to the neighboring cutting turns during longitudinal advancement to thereby increase the cutting area and facilitate insertion of the lead tip into the tissue.

According to a fifth option, which can be combined with any of the first to third options, a conical screw may be fixed at the distal end of the lead tip and surrounded by the fixation helix. The conical screw presses the tissue within the fixation helix outwards to the neighboring cutting turns during longitudinal advancement to thereby increase the cutting area and facilitate insertion of the lead tip into the tissue. Moreover, the conical screw support longitudinal advancement by its thread at the surface.

According to a sixth option, which can be combined with any of the first to third options, the cutting portion may comprise a cutting wire (or other filament or thin bar) arranged between neighboring turns of the fixation helix. During the screwing motion of the fixation helix into the tissue, the tissue is cut by the cutting wire in the circumferential direction as the fixation helix rotates. Thereby, a reliable implementation of the additional cutting effect is obtained.

According to a seventh option, which can be combined with any of the first to third options, the cutting portion may comprise a blade element arranged within the fixation helix. During the screwing motion of the fixation helix into the tissue, the tissue is cut by the inner blade in the circumferential direction of the fixation helix. Thereby, another reliable implementation of the additional cutting effect is obtained.

According to an eighth option, which can be combined with any of the first to sixth options, the lead tip may comprise a first electrode formed by the fixation helix, and an interelectrode portion between the fixation helix and a proximal anode. Thus, the interelectrode portion can be designed according to the requirements of the puncturing process.

According to a ninth option, which can be combined with the eighth option, a ratio between a first outer diameter of the fixation helix and a second outer diameter at a distal end of the interelectrode portion is set between 0.8 and 1, the first outer diameter is set between 1 and 1.8 mm, the length of the lead tip is set between 8 and 15 mm, and the length of the fixation helix is set between 1.5 and 5 mm. Thereby, the dimensions of the lead tip can be designed to facilitate insertion of the lead tip into the tissue during the puncturing process.

According to a tenth option, which can be combined with the eighth or tenth option, the interelectrode portion (between proximal helix end and distal anode end) may have a conical shape. The conical shape facilitates insertion of the lead tip into the tissue during the puncturing process.

According to an eleventh option which can be combined with any of the first to tenth options, the body of the lead device may have a coradial structure. This structure enables a less bulky and stiff lead device, which further facilitates insertion of the lead tip with the fixation helix into the tissue.

According to a twelfth option, which can be combined with any of the first to eleventh options, at least the distal end of the housing of the lead tip, which surrounds the proximal end of the fixation helix, is tapered to obtain a sharpened front end acting as a complementary axial cutting portion. Thereby, the puncturing procedure can be further supported by a complementary cutting effect.

It shall be further understood that a preferred embodiment of the invention can also be any combination of the dependent claims or above embodiments with the respective independent claim.

These and other aspects of the invention will be apparent from and elucidated with reference to the embodiments described hereinafter.

### BRIEF DESCRIPTION OF THE DRAWINGS

In the following drawings:

FIG. 1 shows schematically a heart with a lead device placed for ventricular transseptal LBB pacing;

FIG. 2 shows schematically a side view of a lead tip with conventional fixation helix;

FIG. 3A shows schematically a cross-sectional view of a lead tip with cutting helix according to an embodiment, prior to penetration of the lead tip into the septum;

FIG. 3B shows schematically a cross-sectional view of the lead tip with the cutting helix of FIG. 3A after penetration of the lead tip into the septum;

FIG. 4 shows schematically a side view with partially cut out lead tip housing of a more detailed example of a lead tip with cutting helix;

FIG. 5 shows schematically a cross-sectional view of a lead tip with cutting helix and conical insert according to another embodiment;

FIG. 6 shows schematically a cross-sectional view of a lead tip with cutting helix and conical screw according to a further embodiment;

FIG. 7 shows schematically a cross-sectional view of a lead tip with cutting helix and single-side sharpened cutting portion according to a further embodiment;

FIG. 8 shows schematically a cross-sectional view of a lead tip with cutting helix and double-side sharpened cutting portion according to a further embodiment;

FIG. 9 shows schematically a perspective view of a lead device with a fixation helix comprising a cutting wire according to a further embodiment; and FIG. 10 shows schematically perspective views of a lead device with a fixation helix surrounding an internal cutting blade according to another embodiment.

### DETAILED DESCRIPTION OF EMBODIMENTS

Various embodiments of the present invention are now described based on an improved lead device (e.g., electrode catheter) with fixation helix. Although the present invention is particularly advantageous within the context of transseptal pacing such as LBBP, the invention is not limited thereto and may also be used in connection with other pacing types and/or sites for other applications that require placement of a lead device within a body tissue.

It is noted that throughout the present disclosure only those elements, portions, components and/or devices that are relevant for the proposed lead device and placement operation are shown in the accompanying drawings. Other elements, portions, components and/or devices have been omitted for reasons of brevity. Furthermore, components designated by same reference signs or numbers are intended to have the same or at least a similar function, so that their function is not described again later.

Furthermore, throughout the present disclosure, “proximal” and “distal” are terms that are used to indicate distances from an operating end (reference point) of the lead device, where the physician or other user controls the screwing process. Proximal is closer to the operating end, while distal is further away (at a greater distance) from the operating end.

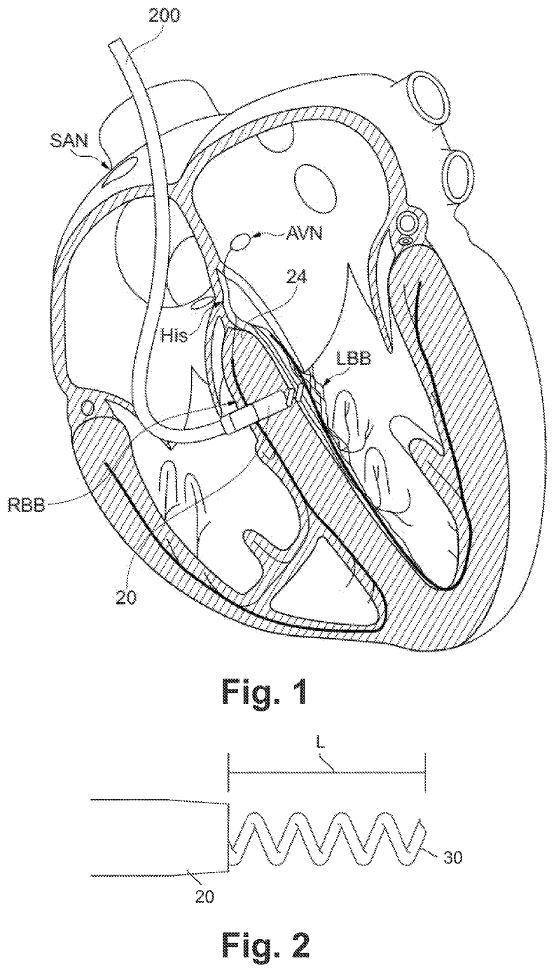

FIG. 1 shows schematically a heart with an inserted lead device 200, where the pacing lead tip 20 is placed for ventricular transseptal LBBP. Thereby, the LV can be paced from the RV by a ventricular transeptal approach as a guide for catheter delivery. The placement of the pacing lead tip 20 may be performed based on the procedure explained above. LBBP may be defined as capture of the LBB (i.e., left bundle trunk or its proximal fascicles), usually with septal myocardium capture at low output (e.g., <1.0 V/0.4 ms).

In a normal cardiac function, the heartbeat starts in the heart itself due to the sinoatrial node (SAN) which is found in the top of the right atrium (RA) and sets the rate at which the heart contracts. It sends out electrical impulses that are carried through the muscular walls of both atria. These impulses cause atrial systole. The impulse is then passed to another node within the heart-the atrioventricular node (AVN). This node is in the lower part of the RA. Once the impulse from the SAN reaches the AVN the impulse is passed to conducting fibers which travel down the central wall of the heart. The impulse then splits and travels up the LV and RV causing them to contract simultaneously (ventricular systole).

Important elements of the conduction system of the heart are found within the septum 24. The His bundle travels in the subendocardium down the right side of the septum 24 for about 1 cm before dividing into the LBB and RBB. The LBB continues down the right side of the septum 24, while the LBB crosses to the left side and splits into anterior and posterior divisions.

Under normal circumstances, excitation from the SAN controls the heart rhythm. An abnormality in the sinus rhythm leads to arrhythmia, which refers to abnormalities in the rate, rhythm, site of origin, and conduction of the cardiac electrical pulse. When disorders occur in specific intraventricular conduction fibers, the repolarization wave must then travel through the slower muscle-muscle conduction to reach the ventricles. Classic disorders related to conditions that involve different conduction bundle branches include LBBB and RBBB. An electrocardiogram (ECG), as obtained from an inserted lead device, can be used to measure and record cardiac electrical activities and thus can provide important information on cardiac functions. The ECG has been used as a standard diagnostic tool to analyze arrhythmia.

The following embodiments of the proposed lead device are configured to minimize any adverse impact of lead device insertion through the septum 24 (e.g., to prevent permanent damages of arteries) by downsizing of the puncturing area and/or introducing a cutting mechanism with an improved fixation helix.

The body of the lead device can be configured to improve slipperiness of a contact with a guiding catheter used for guiding the lead device (e.g., through a blood vessel) to a target area. This can be achieved by using e.g. a polyurethane (PU) material with reduced diameter to allow an advancement progress of the lead body through the guiding catheter and the lead tip 20 through the septum 24 with limited effort.

Suitable designs of the lead device may have a multi-lumen, coaxial and coradial structure, both as tachycardia leads or bradycardia leads, and a central lumen for a stylet passage may be provided. Coaxial leads have an inner conductor that extends down the length of the lead to the tip electrode (helix), the cathode, arranged in a coil configuration that provides a central lumen e.g. to allow for passage of a stylet at implantation.

Coradial bipolar leads address some of the disadvantages of coaxial leads with respect to the bulk and stiffness of their four-layer design, by providing a new conductor and insulator technology where a single coil extends down the length of the lead (again with a central lumen to allow for stylet insertion) and consists of two parallel, alternating conductor strands, one of which connects to the cathode and the other to the anode. Each conductor strand may be individually coated with a bonded layer of e.g. ethylene tetrafluoroethylene (ETFE) fluoropolymer insulation that serves to insulate each strand from the other, despite being intertwined. The single, two-component coil may be surrounded by a single, outer insulation covering.

The multi-lumen or coaxial or coradial leads may optionally comprise a fixed, non-retractable helix to minimize size. However, a retractable helix may also be used in connection with the described embodiments.

Furthermore, the proposed lead system may be configured to provide improved torquability, i.e., an ability to transmit torque safely and accurately to the helix (e.g., full lead body torque) and stylet-driven compatibility to ease the handling (e.g., by push transmission). In an example, a coradial lead with compatible screwing stylet (screwdriver stylet) may be provided.

FIG. 2 shows schematically a side view of a lead tip 20 with conventional fixation helix 30 with an active length L that is screwed into heart tissue solely by puncturing the tissue with the distal tip of the fixation helix 30. The lead device may be used for stimulation of the left and/or right bundle branches. The lead device may for example include an elongate body that extends between a proximal end (not shown) that is configured to interface with an implantable pulse generator and a distal end at the fixation helix 30. The elongate body may also include a lumen that extends between the proximal end and the distal end.

In at least some of the following embodiments, as regards the design of the distal end (distality) of the lead device, the rate between the outer diameter of the helix 30 and the outer diameter of the housing of the lead tip 20 may be larger than 70% ideally 100%, wherein an isoprofil distality may be provided to avoid a front stop surface for better insertion.

Furthermore, the helix 30 may be made of a rigid material to avoid deformation of the helix during screwing, while a fixed helix (i.e., a lock between the helix 30 and the lead body) may simplify handling (i.e., no parasite tool is required for a retractable system).

Moreover, design flexibility may be provided by adapting a distance between the fixation helix and a proximal second cathode for both-side pacing, to be suitable despite different thicknesses of the septum in different people.

The distal design of the lead device may further be configured to allow smooth and predictable advancement of the lead tip 20 into the septum until the helix (cathode) 30 reaches the desired location at the LV chamber, i.e., close to the LBB without full perforation of the septum, so that the helix 30 does not protrude into the LV chamber.

Additionally, the design of the lead device may be configured to minimize the required energy/torque to perform the puncturing of the septum. This may be achieved by providing a dedicated distally tapered lead tip 20 with a conical shape e.g. for the interelectrode section (between proximal helix end and distal anode end).

In certain instances, at least a proximal portion of the fixation helix 30 may be insulated and at least one turn of the distal end of the fixation helix 30 may be uninsulated. One or more turns of the fixation helix 30 (e.g., internal to the lumen of the elongate body) may be covered by a dielectric or other insulative material. The proximal portion or turn of the fixation helix 30 being eliminated may minimize impedance interference that can result from the spacing of a proximal electrode (not shown) and the fixation helix 30.

The fixation helix 30 may be mounted (e.g., welded) on a driver (not shown) which may comprise a surrounding coil or other non-flat regular or irregular surface structure to ensure a good adhesion of the surrounding material of the lead body to the driver at the interelectrode portion (between proximal helix end and distal anode end) to thereby obtain a simple rigid and durable structure of the lead tip 20 with low number of components for improved long-term reliability. The driver may be fixedly supported in a lead body and mechanically and electrically connected to a screwing stylet adapter matched for insertion of a coupling end (engagement portion) of a separate screwing stylet with a screwdriver function for allowing rotational driving of the helix via the driver. Due to an electrical connection between the fixation helix 30 and the screwing stylet, electrical signals sensed by the fixation helix 30 at the target area can be routed via the screwing stylet to a signal analyzer and used for monitoring correct placement of the fixation helix 30 during the screwing operation without any disconnection, allowing a single step operation.

The conical shape of the lead tip 20 may for example be based on dimensional parameters of an outer diameter Da of the proximal anode, an outer diameter Dl of the distal section of the lead tip 20, an outer diameter Dh of the fixation helix 30, the length L of the fixation helix 30, and total length Lt of the lead tip 20 including the fixation helix 30 and a tapered portion of the lead body (e.g., surrounding the driver) between the fixation helix 30 and the proximal anode.

In examples, the rate Dh/Dl can be set between 0.8 and 1, while Dh can be set between 1 and 1.55 mm (preferably 1.40 mm). Da can be set between 1.25 mm and 1.94 mm (preferably 1.66 mm), Lt can be set between 8 and 15 mm, and L can be set between 2 and 5 mm.

This proposed specific conical shape with the above-mentioned ranges of dimension ensures that the lead tip with the fixation helix 30 can be used to puncture tissue in the target area in a controlled and smooth manner providing a conic profile minimizing the required energy to perform the puncture.

In the following embodiments, the structure of the fixation helix is enhanced by providing an additional cutting function for cutting the tissue in a circumferential direction of the helix, when torque is applied to the lead tip, to facilitate and smoothen the translation (longitudinal movement) of the lead tip with the helix during the rotational puncturing process. Thereby, a cutting helix is obtained. More specifically, a cutting portion or element of or at the cutting helix is configured to locally dissect the tissue located within, at or around the cutting helix and/or expand the tissue opening for the lead tip body during the rotational and longitudinal movement of the cutting helix into the tissue. As a result, the rotational drilling or puncturing process by the tip at the distal end of the cutting helix is supported by an additional circumferential cutting process when the cutting helix is screwed into the tissue, so that the energy/torque required to perform the puncturing of the tissue can be reduced substantially.

In an embodiment, the cutting portion may be created by reducing or closing the distance or width of the longitudinal gap section between neighboring turns at the proximal portion of the cutting helix. During screwing of the helix into the tissue, rotation is transformed into longitudinal advancement of the lead tip into the tissue. The helix motricity (longitudinal advancement by screwing) for tip advancement is preserved, as the cutting effect by the reduced or closed gap portion is provided at a portion (e.g., proximal end) of the helix, which is more proximal than the helix motricity section. The reduced or closing gap section of the helix acts as rotational pair of scissors. This section is exposed to the engaged tissue, which it cuts locally, as a result of the rotational movement of the lead tip during screwing of the helix into the tissue.

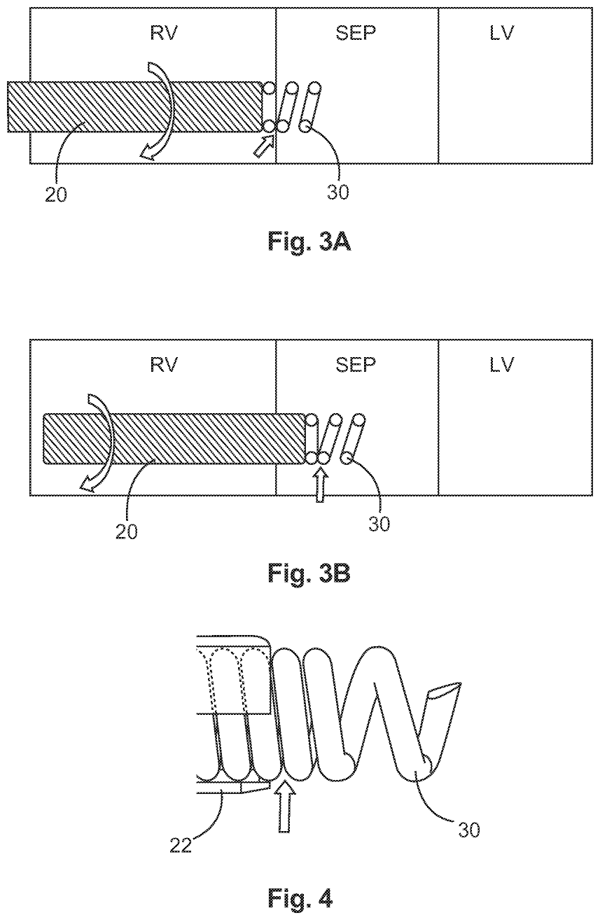

FIG. 3A shows schematically a cross-sectional view of a lead tip 20 with cutting helix 30 according to an embodiment, prior to penetration of the lead tip 20 from the RV into the septum (SEP). The rotational screwing motion is indicated by the half-circular arrow around the lead tip 20. Conventionally, the screwing motion is clockwise for tissue engagement and attachment. Furthermore, the straight arrow at the cutting helix 30 points towards the cutting or dissection portion of the cutting helix, where the gap width between neighboring turns of the helix is reduced e.g. to zero. The distal portion of the cutting helix 30 with the conventional gap width forms the motricity section.

When the helix 30 is screwed further into the tissue, the tissue grabbed by distal portion (motricity section) of the cutting helix 30 will be moved towards the cutting or dissection section of the cutting helix 30, where the grabbed tissue will be exposed to high axial pressure between rigid (substantially non-deformable metal wire sections) when the gap width decreases, effectively resulting in a tissue-cutting process.

FIG. 3B shows schematically a cross-sectional view of the lead tip 20 with the cutting helix 30 of FIG. 3A after penetration of the lead tip 20 into the septum. The penetration of the lead tip 20 into the septum is facilitated due to the additional/supportive circular cutting of the tissue at the circumference of the cutting helix 30.

The supportive cutting process will be continued during the whole puncture process until the physician stops rotation of the lead device, either at the proximal end, or if a stylet is used, at a driver which may be provided at the distal end.

FIG. 4 shows schematically a side view of a more detailed example of a lead tip 20 with cutting helix 30 with partially cut out lead tip housing 22. The lead tip housing 22 may be designed with a specific conical shape, e.g., with the dimensions described above, or with any other shape suitable for the puncturing process. Thus, the lead tip design of FIG. 4 may be used for LBBP-type of applications.

The distal end of the housing 22 may optionally be tapered to obtain a thinned or sharpened front end, acting as a complementary axial cutting portion (e.g., scissor). The housing 22 also covers a proximal section of the cutting helix 30 (where a driver (not shown) may be attached) to limit the total electrical surface of the cutting helix 30 (e.g., to preserve electrical performance). The housing 22 could be made with plastic material like polyetheretherketon (PEEK) due to its high biocompatible properties and extremely rigid mechanical structure to support the cutting effect without any risk of local deformation.

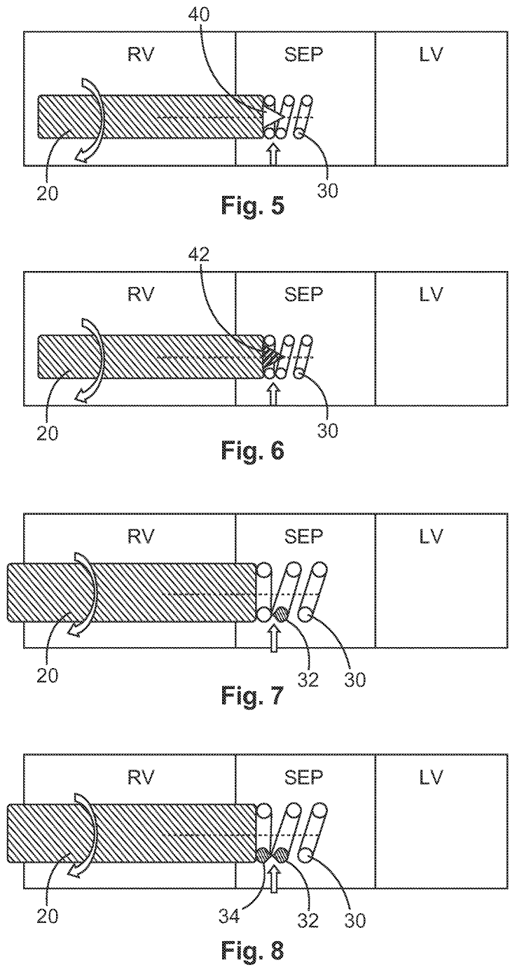

FIG. 5 shows schematically a cross-sectional view of a lead tip 20 with cutting helix 30 and conical insert 40 according to another embodiment.

The conical insert 40 may be removably or non-removably fixed (e.g., screwed, glued, press-fitted, molded, etc.) to the lead tip 30 within the cutting helix 30 and made of a metal or other rigid material to support the cutting process by forcing the tissue within the cutting helix 30 to move out radially toward the cutting portion (rotational scissor).

The conical insert 40 may be rotationally symmetrical, which facilitates its manufacturing.

FIG. 6 shows schematically a cross-sectional view of a lead tip 20 with cutting helix 30 and conical screw 42 according to a further embodiment. Similar to the conical insert of FIG. 5, the conical screw 42 may be made of a metal or other rigid material. In addition to the tissue directing function, the conical screw 42 provides an axial motion function that support the motricity portion of the cutting helix 30.

The conical screw 42 could be manufactured by cutting a thread into the conical insert 40 of FIG. 5 or by otherwise machining a local thin conical screw (filets) onto the conical insert 40.

FIG. 7 shows schematically a cross-sectional view of a lead tip 20 with cutting helix 30 and single-side sharpened cutting portion according to a further embodiment.

In the embodiment of FIG. 7, the cross-sectional profile or shape of a cutting turn 32 of the cutting helix 30 at the minimal-gap or gapless cutting portion (i.e., where the gap between the neighboring turns is closed) is modified to provide a sharp edge facing the closed neighboring turn to thereby increase the sharpness of the scissor-like cutting portion to locally improve the cutting effect.

The sharpened shape of the cutting turn 32 may be mainly oriented in an axial direction. The sharpened design may be achieved by locally forming a sharpened section of a wire and later forming the wire into a coiled shape to obtain the cutting helix 30 with the cutting turn 32.

FIG. 8 shows schematically a cross-sectional view of a lead tip 20 with cutting helix 30 and double-side sharpened cutting portion with neighboring cutting turns 32, 34 having opposed sharp edges according to a further embodiment.

With the neighboring cutting turns 32, 34, two opposite sharpened profiles face each other to thereby further increase the cutting pressure against the tissue during the screwing process.

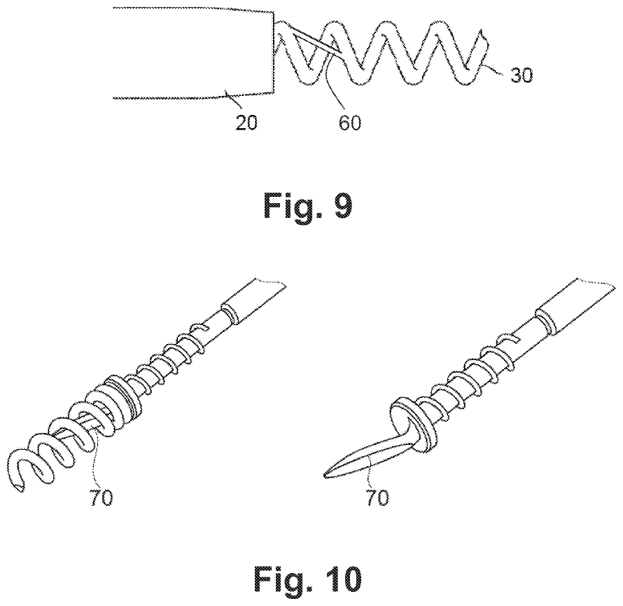

FIG. 9 shows schematically a perspective view of a lead device according to a further embodiment, where the cutting function or cutting portion of the cutting helix is achieved by a thin wire 60 (or other filament or thin bar) that may be welded between two proximal windings of the cutting helix in order to locally dissect the tissue when the helix is screwed into the tissue.

FIG. 10 shows schematically perspective views of a lead device according to another embodiment, where the cutting function or cutting portion of the cutting helix is achieved by an additional cutting blade 70 at the lead tip of the lead device. For better indication of the shape of the cutting blade 70, the right part of FIG. 10 shows a disassembled configuration of the lead device with removed helix.

The additional cutting blade 70 is configured to minimize the required energy/force to perform the puncturing of the tissue of the septum by locally dissecting the tissue located within the helix and thereby expanding the tissue opening for the lead body during the rotational and longitudinal movement of the cutting helix into the tissue. The oval shape of the blade 70 may be configured to have a sharp(er) side edge in the direction of rotation to cut through the tissue. However, any other shape (e.g., triangular, rectangular, etc.) of the blade 70 may be provided. In the configuration of FIG. 10, the blade 70 is protected inside the helix.

As an alternative, (an)other cutting element(s) with other shapes may be provided around or within the helix.

In all above embodiments of FIGS. 3 to 10, the special puncture design (e.g., conical shape) of the lead tip 20 with the dimensions described above can be applied.

To summarize, a lead device that comprises a lead tip with a distal fixation helix for fixing the lead tip at a patient's tissue has been described, wherein the fixation helix comprises a cutting portion (exposed to the tissue) for cutting the tissue in a circumferential direction of the fixation helix in addition to the puncturing by the distal tip of the fixation helix, when the fixation helix is screwed into the tissue, to support longitudinal insertion of the lead tip with the fixation helix into the tissue.

While the invention has been illustrated and described in detail in the drawings and foregoing description, such illustration and description are to be considered illustrative or exemplary and not restrictive. The invention is not limited to the disclosed embodiments. It can be applied to various types of lead devices (e.g., bradycardia or tachycardia lead devices with multi-lumen, coaxial or coradial structure) and applications in the field of cardiac pacing or sensing systems.

The proposed lead device with cutting helix may be configured to be adapted or adaptable to IS1, IS4 (low voltage) or DF4 (high voltage) connectors.

As another option, the cutting helix 30 of FIGS. 3, 4, 7 and 8 may be combined with the additional cutting wire 60 of FIG. 9 and/or the additional blade 70 of FIG. 10 at the tip of the lead device.

Other variations to the disclosed embodiments can be understood and effected by those skilled in the art in practicing the claimed invention, from a study of the drawings, the disclosure and the appended claims. In the claims, the word “comprising” does not exclude other elements or steps, and the indefinite article “a” or “an” does not exclude a plurality. The mere fact that certain measures are recited in mutually different dependent claims does not indicate that a combination of these measures cannot be used to advantage. The foregoing description details certain embodiments of the invention. It will be appreciated, however, that no matter how detailed the foregoing appears in the text, the invention may be practiced in many ways, and is therefore not limited to the embodiments disclosed. It should be noted that the use of particular terminology when describing certain features or aspects of the invention should not be taken to imply that the terminology is being re-defined herein to be restricted to include any specific characteristics of the features or aspects of the invention with which that terminology is associated.

1. A lead device comprising a lead tip with a distal fixation helix for fixing the lead tip at a patient's tissue, wherein the fixation helix comprises a cutting portion for cutting the tissue in a circumferential direction of the fixation helix in addition to a puncturing by a distal tip of the fixation helix, when the fixation helix is screwed into the tissue, to support longitudinal insertion of the lead tip with the fixation helix into the tissue.

2. The lead device of claim 1, wherein the cutting portion comprises two exposed neighboring turns of the fixation helix with a closed intermediate gap in the longitudinal direction between the two neighboring turns.

3. The lead device of claim 2, wherein the two neighboring turns of the fixation helix are provided in a proximal portion of the fixation helix.

4. The lead device of claim 2, wherein at least one of the two neighboring turns of the fixation helix comprises a cross-sectional shape with a sharp edge towards the opposing neighboring turn.

5. The lead device of claim 2, further comprising a conical insert fixed at the distal end of the lead tip and surrounded by the fixation helix.

6. The lead device of claim 2, further comprising a conical screw fixed at the distal end of the lead tip and surrounded by the fixation helix.

7. The lead device of claim 1, wherein the cutting portion comprises a blade element arranged within the fixation helix.

8. The lead device of claim 1, wherein the cutting portion comprises a cutting wire arranged between neighboring turns of the fixation helix.

9. The lead device of claim 1, wherein the lead tip comprises a first electrode formed by the fixation helix, and an interelectrode portion between the fixation helix and a proximal anode.

10. The lead device of claim 9, wherein a ratio between a first outer diameter of the fixation helix and a second outer diameter at a distal end of the interelectrode portion is set between 0.8 and 1, the first outer diameter is set between 1 and 1.8 mm, the length of the lead tip is set between 8 and 15 mm, and the length of the fixation helix is set between 1.5 and 5 mm.

11. The lead device of claim 9, wherein the interelectrode portion has a conical shape.

12. The lead device of claim 1, wherein the body of the lead device has a coradial structure.

13. The lead device of claim 1, wherein at least the distal end of the housing of the lead tip, that surrounds the proximal end of the fixation helix, is tapered to obtain a sharpened front end acting as a complementary axial cutting portion.
